(19) United States
(12) Patent Application Publication
(10) Pub. No.: US 2026/0124220 A1
(43) Pub. Date: May 7, 2026

(54) COMPOSITIONS AND METHODS RELATED TO ISOMERS OF CURCUMIN

(71) Applicant: Natural Extraction Systems, LLC, Boulder, CO (US)

(72) Inventors: C. Russell Thomas, Boulder, CO (US); Douglas G. Metcalf, Pilot Mountain, NC (US)

(21) Appl. No.: 19/438,983

(22) Filed: Jan. 2, 2026

Related U.S. Application Data
(62) Division of application No. 17/751,409, filed on May 23, 2022, now Pat. No. 12,514,864.
(60) Provisional application No. 63/191,818, filed on May 21, 2021.
(60) Provisional application No. 63/191,836, filed on May 21, 2021.
(60) Provisional application No. 63/191,849, filed on May 21, 2021.
(60) Provisional application No. 63/191,880, filed on May 21, 2021.
(60) Provisional application No. 63/194,815, filed on May 28, 2021.
(60) Provisional application No. 63/254,437, filed on Oct. 11, 2021.

(51) Int. Cl. A61K 31/00; A61K 9/00; A61K 31/05; A61K 31/352; A61K 31/538; A61K 47/10

(52) U.S. Cl. CPC A61K 31/658; A61K 9/0053; A61K 31/05; A61K 31/352; A61K 31/538; A61K 47/10

(57) ABSTRACT

Various aspects of this disclosure relate to compositions comprising anions produced from curcumin, containers containing compositions comprising anions produced from curcumin, and methods of using compositions comprising anions produced from curcumin. Without limiting this disclosure or any patent claim that matures from this disclosure, anions produced from curcumin display improved bioavailability relative to molecular curcumin.

### CROSS-REFERENCE TO RELATED APPLICATIONS

This application is a Division of U.S. patent application Ser. No. 17/751,409, which granted as U.S. Pat. No. 12,514,864, and claims priority to U.S. Provisional Patent Application No. 63/191,818, filed May 21, 2021, U.S. Provisional Patent Application No. 63/191,836, filed May 21, 2021, U.S. Provisional Patent Application No. 63/191,849, filed May 21, 2021, U.S. Provisional Patent Application No. 63/191,880, filed May 21, 2021, U.S. Provisional Patent Application No. 63/194,815, filed May 28, 2021, and U.S. Provisional Patent Application No. 63/254,437, filed Oct. 11, 2021, each of which is incorporated by reference in its entirety.

### BACKGROUND

Curcumin is the major bioactive molecule of turmeric, which is an ingredient and dietary supplement. Curcumin is known to be effective at treating cancer and inflammation. Numerous attempts to develop curcumin pharmaceuticals nevertheless failed in part because curcumin has limited solubility and limited bioavailability and because curcumin is thought to have limited chemical stability. Curcumin formulations with improved solubility, bioavailability, and stability are desirable.

### SUMMARY

Various aspects of this disclosure relate to anionic forms of structural isomers of curcumin.

Without limiting this disclosure or any patent claim that matures from this document, anionic forms of curcumin and its structural isomers display improved solubility in water relative to molecular forms because anionic forms carry negative charges, which favors hydrogen bonding to water; because charge-charge repulsion maximizes surface area in liquid formulations, which generally improves bioavailability and pharmacokinetics; because the administration of anionic forms of curcumin and its structural isomers to subjects converts the anionic forms to molecular forms, which partition out of liquid formulations and into the epithelial lining of a subject for rapid absorption, which improves bioavailability and pharmacokinetics; and because the molecular forms avoid first-pass metabolism, for example, when the molecular forms partition into the epithelial lining of the mouth or esophagus, which improves bioavailability and pharmacokinetics.

Without limiting this disclosure or any patent claim that matures from this document, those skilled in the relevant arts generally believed that liquid formulations comprising curcumin generally, and the anionic forms of curcumin specifically, lacked sufficient stability to be pharmaceutically relevant; the perceived instability of curcumin generally, and the anionic forms of curcumin specifically, arose in part from the conversion of curcumin into one or more structural isomers of curcumin, which decreases measured curcumin concentrations in curcumin formulations.

This patent document discloses anionic forms of structural isomers of curcumin that can be manufactured from curcumin using acid-base chemistry. The anionic forms of structural isomers of curcumin display improved solubility, bioavailability, and pharmacokinetics relative to known curcumin formulations, and they are expected to display comparable or improved efficacy at treating the health conditions that curcumin is known to treat.

Various aspects of this disclosure relate to structural isomers of curcumin and their conjugate bases. Curcuma longa (turmeric) biosynthetically produces curcumin, which is a tautomer that exists as the enol structure of Compound I and the keto structure of Compound II.

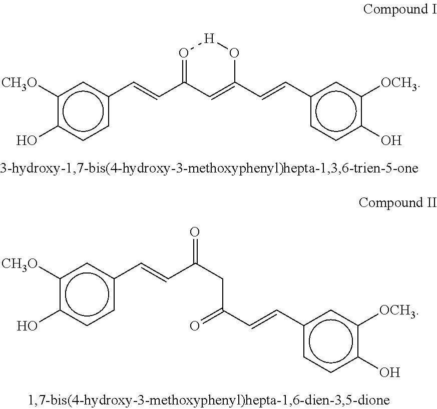

The molecule of Compound I can be deprotonated to produce the anions of Compounds III and IV.

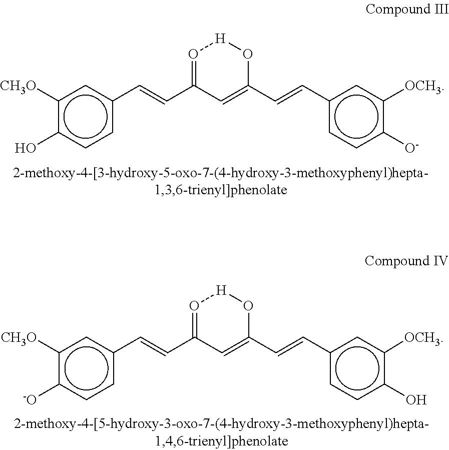

The molecule of Compound II can be deprotonated to produce the anion of Compound V.

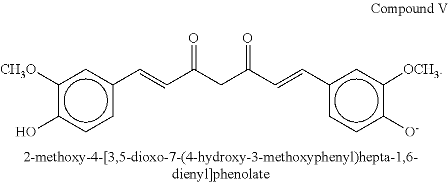

The anion of Compound III has the resonance structures of Compounds VI, VII, and VIII.

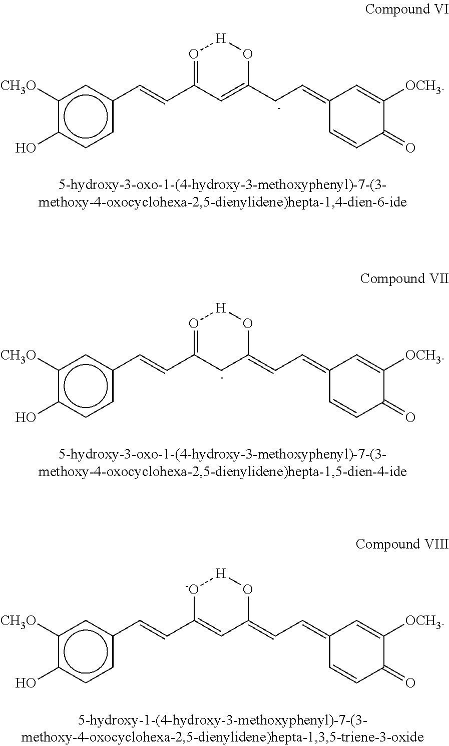

The anion of Compound VI can be reprotonated to produce the molecule of Compound IX.

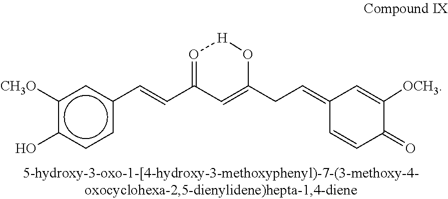

The anion of Compound VII can be reprotonated to produce the molecule of Compound X.

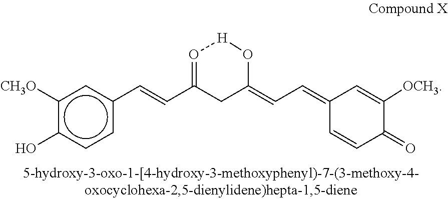

The anion of Compound VIII can be reprotonated to produce the molecule of Compound XI.

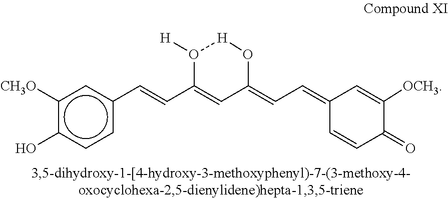

The anion of Compound IV has the resonance structure of Compound XII.

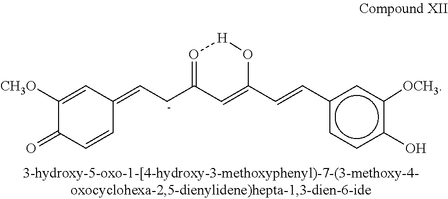

The anion of Compound XII can be reprotonated to produce the molecule of Compound XIII.

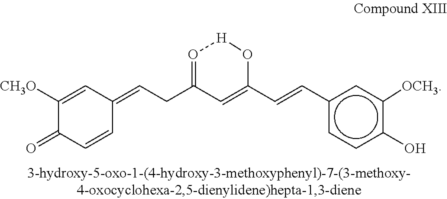

The anion of Compound V has the resonance structure of Compound XIV.

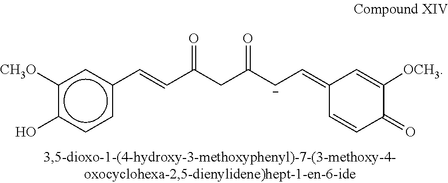

The anion of Compound XIV can be reprotonated to produce the molecule of Compound XV.

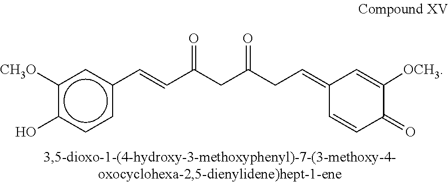

The molecule of Compound IX can be deprotonated to produce the anion of Compound XVI.

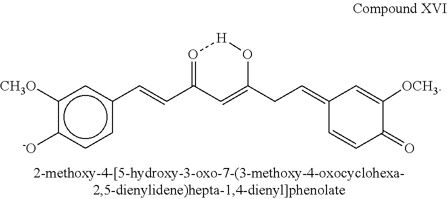

The anion of Compound XVI has the resonance structures of Compounds XVII and XVIII.

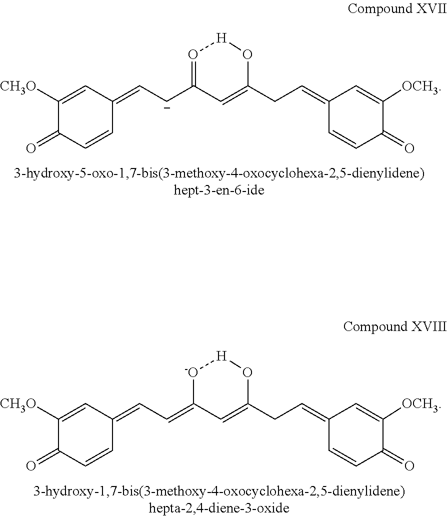

The anion of Compound XVII can be reprotonated to produce the molecule of Compound XIX.

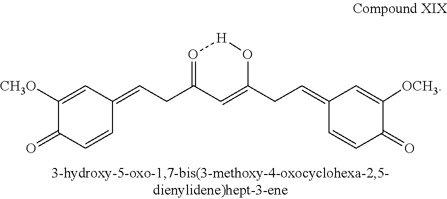

The anion of Compound XVIII can be reprotonated to produce the molecule of Compound XX.

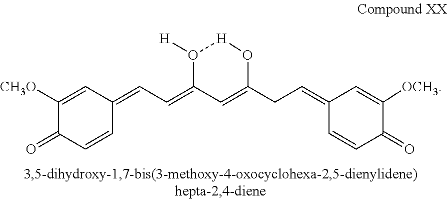

The molecule of Compound X can be deprotonated to produce the anion of Compound XXI.

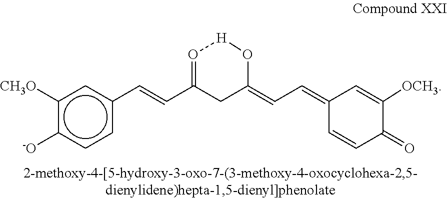

The anion of Compound XXI has the resonance structures of Compounds XXII and XXIII.

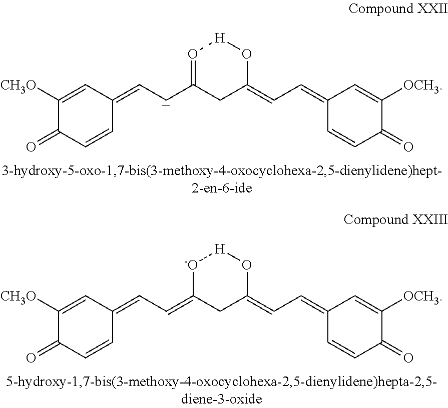

The anion of Compound XXII can be reprotonated to produce the molecule of Compound XXIV.

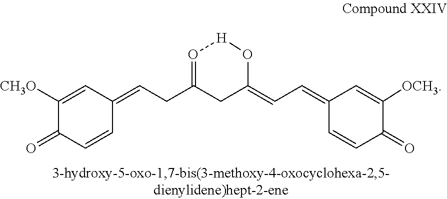

The anion of Compound XXIII can be reprotonated to produce the molecule of Compound XXV.

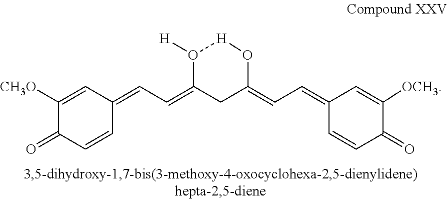

The molecule of Compound XV can be deprotonated to produce the anion of Compound XXVI.

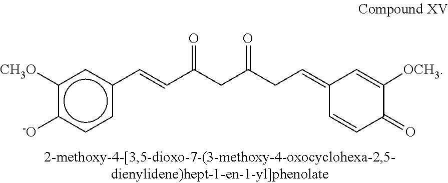

The anion of Compound XXVI has the resonance structure of Compound XXVII.

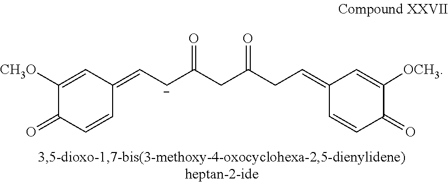

The anion of Compound XXVII can be reprotonated to produce the molecule of Compound XXVIII.

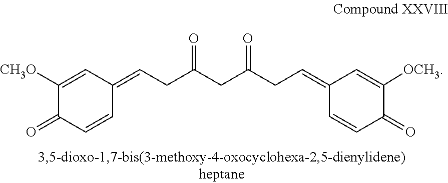

Compounds IX, X, XI, XIII, XV, XIX, XX, XXIV, XXV, and XXVIII depict the major molecular products that can be produced from curcumin using acid-base chemistry in a protic polar solvent such as ethanol. Compounds III, IV, V, VI, VII, VIII, XII, XIV, XVI, XVII, XVIII, XXI, XXII, XXIII, XXVI, and XXVII depict a subset of the major anion products that can be produced from curcumin using acid-base chemistry in a protic polar solvent, and additional major anion products are described in the detailed description that follows.

### DETAILED DESCRIPTION

Various aspects of this disclosure relate to a composition, comprising (i) an anion, in which the anion has the chemical formula C₂₁H₁₉O₆¹⁻; the anion has the chemical structure set forth in Compound XXIX; Compound XXIX contains exactly 10 double bonds; R1 is hydroxy, oxide, or oxo; R2 is hydroxy or oxo; and the 4 dotted lines in Compound XXIX depict 0, 1, or 2 double bonds, (ii) a cation at a concentration of at least 10 nanomolar and no greater than 1 molar, in which the cation is a metal cation or an ammonium cation, and (iii) a protic polar solvent at a concentration of at least 5 molar and no greater than 55.5 molar, in which the anion and the cation are solutes that are dissolved in the protic polar solvent.

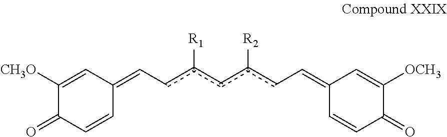

Each “hydroxy” and each “oxide” contributes exactly 0 double bonds by definition. Each “oxo” contributes exactly 1 double bond by definition. Each of the 21 carbon atoms participates in either exactly 0 or exactly 1 double bond. The carbon atom that is covalently bound to R1 participates in exactly 1 double bond. The carbon atom that is covalently bound to R2 participates in exactly 1 double bond.

“Comprise” refers to an open set such that a composition that comprises an anion, a cation, and a solvent can also comprise a molecule, a Brønsted base, and a cosolvent.

“In which” is an exact synonym of “wherein.”

“Ammonium cation” refers to either ammonium (“NH4+”) or an aminium cation.

“Dissolved” refers to a chemical species that is solvated by either (i) a solvent, (ii) a cosolvent, or (iii) both a solvent and a cosolvent; a chemical species that is dispersed within a solvent, such as the dispersed phase of an emulsion, is not dissolved; a chemical species that is non-covalently bound to any chemical species that is a solid in the absence of a solvent, such as a cyclodextrin, is not dissolved.

The following paragraph enumerates various anionic species of Compound XXIX.

In some embodiments, the anion is either 5-hydroxy-1,7-bis(3-methoxy-4-oxocyclohexa-2,5-dienylidene)hepta-2,4-diene-3-oxide; 5-hydroxy-1,7-bis(3-methoxy-4-oxocyclohexa-2,5-dienylidene)hepta-2,5-diene-3-oxide; 5-hydroxy-1,7-bis(3-methoxy-4-oxocyclohexa-2,5-dienylidene)hepta-3,5-diene-3-oxide; 5-oxo-1,7-bis(3-methoxy-4-oxocyclohexa-2,5-dienylidene)hept-2-ene-3-oxide; 5-oxo-1,7-bis(3-methoxy-4-oxocyclohexa-2,5-dienylidene)hept-3-ene-3-oxide; 3-hydroxy-5-oxo-1,7-bis(3-methoxy-4-oxocyclohexa-2,5-dienylidene)hept-2-en-4-ide; 3-hydroxy-5-oxo-1,7-bis(3-methoxy-4-oxocyclohexa-2,5-dienylidene)hept-2-en-6-ide; 3-hydroxy-5-oxo-1,7-bis(3-methoxy-4-oxocyclohexa-2,5-dienylidene)hept-3-en-2-ide; 3-hydroxy-5-oxo-1,7-bis(3-methoxy-4-oxocyclohexa-2,5-dienylidene)hept-3-en-6-ide; 3,5-dioxo-1,7-bis(3-methoxy-4-oxocyclohexa-2,5-dienylidene)heptan-2-ide; 3,5-dioxo-1,7-bis(3-methoxy-4-oxocyclohexa-2,5-dienylidene)heptan-4-ide; 3,5-dihydroxy-1,7-bis(3-methoxy-4-oxocyclohexa-2,5-dienylidene)hepta-2,5-dien-4-ide; or 3,5-dihydroxy-1,7-bis(3-methoxy-4-oxocyclohexa-2,5-dienylidene)hepta-2,4-dien-6-ide. The presence of an anion in a composition can be detected, for example, by converting the anion into its conjugate acid and then identifying the conjugate acid using conventional analytical methods such as HPLC-MS.

In some embodiments, the anion is 5-hydroxy-1,7-bis(3-methoxy-4-oxocyclohexa-2,5-dienylidene)hepta-2,4-diene-3-oxide, and the composition further comprises 3,5-dihydroxy-1,7-bis(3-methoxy-4-oxocyclohexa-2,5-dienylidene)hepta-2,4-diene (which is the conjugate acid of 5-hydroxy-1,7-bis(3-methoxy-4-oxocyclohexa-2,5-dienylidene)hepta-2,4-diene-3-oxide).

In some embodiments, the anion is 5-hydroxy-1,7-bis(3-methoxy-4-oxocyclohexa-2,5-dienylidene)hepta-2,5-diene-3-oxide, and the composition further comprises 3,5-dihydroxy-1,7-bis(3-methoxy-4-oxocyclohexa-2,5-dienylidene)hepta-2,5-diene (which is the conjugate acid of 5-hydroxy-1,7-bis(3-methoxy-4-oxocyclohexa-2,5-dienylidene)hepta-2,5-diene-3-oxide).

In some embodiments, the anion is 5-hydroxy-1,7-bis(3-methoxy-4-oxocyclohexa-2,5-dienylidene)hepta-3,5-diene-3-oxide, and the composition further comprises 3,5-dihydroxy-1,7-bis(3-methoxy-4-oxocyclohexa-2,5-dienylidene)hepta-2,4-diene (which is the conjugate acid of 5-hydroxy-1,7-bis(3-methoxy-4-oxocyclohexa-2,5-dienylidene)hepta-3,5-diene-3-oxide).

In some embodiments, the anion is 5-oxo-1,7-bis(3-methoxy-4-oxocyclohexa-2,5-dienylidene)hept-2-ene-3-oxide, and the composition further comprises 3-hydroxy-5-oxo-1,7-bis(3-methoxy-4-oxocyclohexa-2,5-dienylidene)hept-2-ene (which is the conjugate acid of 5-oxo-1,7-bis(3-methoxy-4-oxocyclohexa-2,5-dienylidene)hept-2-ene-3-oxide).

In some embodiments, the anion is 5-oxo-1,7-bis(3-methoxy-4-oxocyclohexa-2,5-dienylidene)hept-3-ene-3-oxide, and the composition further comprises 3-hydroxy-5-oxo-1,7-bis(3-methoxy-4-oxocyclohexa-2,5-dienylidene)hept-3-ene (which is the conjugate acid of 5-oxo-1,7-bis(3-methoxy-4-oxocyclohexa-2,5-dienylidene)hept-3-ene-3-oxide).

In some embodiments, the anion is 3-hydroxy-5-oxo-1,7-bis(3-methoxy-4-oxocyclohexa-2,5-dienylidene)hept-2-en-4-ide, and the composition further comprises 3-hydroxy-5-oxo-1,7-bis(3-methoxy-4-oxocyclohexa-2,5-dienylidene)hept-2-ene (which is the conjugate acid of 3-hydroxy-5-oxo-1,7-bis(3-methoxy-4-oxocyclohexa-2,5-dienylidene)hept-2-en-4-ide).

In some embodiments, the anion is 3-hydroxy-5-oxo-1,7-bis(3-methoxy-4-oxocyclohexa-2,5-dienylidene)hept-2-en-6-ide, and the composition further comprises 3-hydroxy-5-oxo-1,7-bis(3-methoxy-4-oxocyclohexa-2,5-dienylidene)hept-2-ene (which is the conjugate acid of 3-hydroxy-5-oxo-1,7-bis(3-methoxy-4-oxocyclohexa-2,5-dienylidene)hept-2-en-6-ide).

In some embodiments, the anion is 3-hydroxy-5-oxo-1,7-bis(3-methoxy-4-oxocyclohexa-2,5-dienylidene)hept-3-en-2-ide, and the composition further comprises 3-hydroxy-5-oxo-1,7-bis(3-methoxy-4-oxocyclohexa-2,5-dienylidene)hept-3-ene (which is the conjugate acid of 3-hydroxy-5-oxo-1,7-bis(3-methoxy-4-oxocyclohexa-2,5-dienylidene)hept-3-en-2-ide).

In some embodiments, the anion is 3-hydroxy-5-oxo-1,7-bis(3-methoxy-4-oxocyclohexa-2,5-dienylidene)hept-3-en-6-ide, and the composition further comprises 3-hydroxy-5-oxo-1,7-bis(3-methoxy-4-oxocyclohexa-2,5-dienylidene)hept-3-ene (which is the conjugate acid of 3-hydroxy-5-oxo-1,7-bis(3-methoxy-4-oxocyclohexa-2,5-dienylidene)hept-3-en-6-ide).

In some embodiments, the anion is 3,5-dioxo-1,7-bis(3-methoxy-4-oxocyclohexa-2,5-dienylidene)heptan-2-ide, and the composition further comprises 3,5-dioxo-1,7-bis(3-methoxy-4-oxocyclohexa-2,5-dienylidene)heptane (which is the conjugate acid of 3,5-dioxo-1,7-bis(3-methoxy-4-oxocyclohexa-2,5-dienylidene)heptan-2-ide).

In some embodiments, the anion is 3,5-dioxo-1,7-bis(3-methoxy-4-oxocyclohexa-2,5-dienylidene)heptan-4-ide, and the composition further comprises 3,5-dioxo-1,7-bis(3-methoxy-4-oxocyclohexa-2,5-dienylidene)heptane (which is the conjugate acid of 3,5-dioxo-1,7-bis(3-methoxy-4-oxocyclohexa-2,5-dienylidene)heptan-4-ide).

In some embodiments, the anion is 3,5-dihydroxy-1,7-bis(3-methoxy-4-oxocyclohexa-2,5-dienylidene)hepta-2,5-dien-4-ide, and the composition further comprises 3,5-dihydroxy-1,7-bis(3-methoxy-4-oxocyclohexa-2,5-dienylidene)hepta-2,5-diene (which is the conjugate acid of 3,5-dihydroxy-1,7-bis(3-methoxy-4-oxocyclohexa-2,5-dienylidene)hepta-2,5-dien-4-ide).

In some embodiments, the anion is 3,5-dihydroxy-1,7-bis(3-methoxy-4-oxocyclohexa-2,5-dienylidene)hepta-2,4-dien-6-ide, and the composition further comprises 3,5-dihydroxy-1,7-bis(3-methoxy-4-oxocyclohexa-2,5-dienylidene)hepta-2,4-diene (which is the conjugate acid of 3,5-dihydroxy-1,7-bis(3-methoxy-4-oxocyclohexa-2,5-dienylidene)hepta-2,4-dien-6-ide).

Various aspects of this disclosure relate to a composition, comprising (i) an anion, in which the anion has the chemical formula C₂₁H₁₉O₆¹⁻; the anion has the chemical structure set forth in Compound XXX; Compound XXX contains exactly 10 double bonds; R1 is hydroxy, oxide, or oxo; R2 is hydroxy, oxide, or oxo; R3 is hydroxy or oxide; and the 3 dotted lines in Compound XXX depict 0, 1, or 2 double bonds, (ii) a cation at a concentration of at least 10 nanomolar and no greater than 1 molar, in which the cation is a metal cation or an ammonium cation, and (iii) a protic polar solvent at a concentration of at least 5 molar and no greater than 55.5 molar, in which the anion and the cation are solutes that are dissolved in the protic polar solvent.

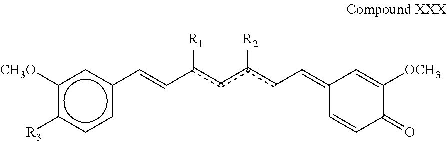

Each “hydroxy” and each “oxide” contributes exactly 0 double bonds by definition. Each “oxo” contributes exactly 1 double bond by definition. Each of the 21 carbon atoms participates in either exactly 0 or exactly 1 double bond. The carbon atom that is covalently bound to R1 participates in exactly 1 double bond. The carbon atom that is covalently bound to R2 participates in exactly 1 double bond. The following paragraph enumerates various anionic species of Compound XXX.

In some embodiments, the anion is either 2-methoxy-4-[3-hydroxy-5-oxo-7-(3-methoxy-4-oxocyclohexa-2,5-dienylidene)hepta-1,3-dienyl]phenolate; 2-methoxy-4-[5-hydroxy-3-oxo-7-(3-methoxy-4-oxocyclohexa-2,5-dienylidene)hepta-1,4-dienyl]phenolate; 2-methoxy-4-[5-hydroxy-3-oxo-7-(3-methoxy-4-oxocyclohexa-2,5-dienylidene)hepta-1,5-dienyl]phenolate; 2-methoxy-4-[3,5-dioxo-7-(3-methoxy-4-oxocyclohexa-2,5-dienylidene)hept-1-en-1-yl]phenolate; 2-methoxy-4-[3,5-dihydroxy-7-(3-methoxy-4-oxocyclohexa-2,5-dienylidene)hepta-1,3,5-trienyl]phenolate; 5-hydroxy-1-(4-hydroxy-3-methoxyphenyl)-7-(3-methoxy-4-oxocyclohexa-2,5-dienylidene)hepta-1,3,5-triene-3-oxide; 3-hydroxy-1-(4-hydroxy-3-methoxyphenyl)-7-(3-methoxy-4-oxocyclohexa-2,5-dienylidene)hepta-1,3,5-triene-5-oxide; 5-oxo-1-(4-hydroxy-3-methoxyphenyl)-7-(3-methoxy-4-oxocyclohexa-2,5-dienylidene)hepta-1,3-diene-3-oxide; 3-oxo-1-(4-hydroxy-3-methoxyphenyl)-7-(3-methoxy-4-oxocyclohexa-2,5-dienylidene)hepta-1,4-diene-5-oxide; 3-oxo-1-(4-hydroxy-3-methoxyphenyl)-7-(3-methoxy-4-oxocyclohexa-2,5-dienylidene)hepta-1,5-diene-5-oxide; 3-hydroxy-5-oxo-1-(4-hydroxy-3-methoxyphenyl)-7-(3-methoxy-4-oxocyclohexa-2,5-dienylidene)hepta-1,3-dien-6-ide; 5-hydroxy-3-oxo-1-(4-hydroxy-3-methoxyphenyl)-7-(3-methoxy-4-oxocyclohexa-2,5-dienylidene)hepta-1,4-dien-6-ide; 5-hydroxy-3-oxo-1-(4-hydroxy-3-methoxyphenyl)-7-(3-methoxy-4-oxocyclohexa-2,5-dienylidene)hepta-1,5-dien-4-ide; 3,5-dioxo-1-(4-hydroxy-3-methoxyphenyl)-7-(3-methoxy-4-oxocyclohexa-2,5-dienylidene)hept-1-en-4-ide; or 3,5-dioxo-1-(4-hydroxy-3-methoxyphenyl)-7-(3-methoxy-4-oxocyclohexa-2,5-dienylidene)hept-1-en-6-ide.

In some embodiments, the anion is 2-methoxy-4-[3-hydroxy-5-oxo-7-(3-methoxy-4-oxocyclohexa-2,5-dienylidene)hepta-1,3-dienyl]phenolate, and the composition further comprises 3-hydroxy-5-oxo-1-(4-hydroxy-3-methoxyphenyl)-7-(3-methoxy-4-oxocyclohexa-2,5-dienylidene)hepta-1,3-diene (which is the conjugate acid of 2-methoxy-4-[3-hydroxy-5-oxo-7-(3-methoxy-4-oxocyclohexa-2,5-dienylidene)hepta-1,3-dienyl]phenolate).

In some embodiments, the anion is 2-methoxy-4-[5-hydroxy-3-oxo-7-(3-methoxy-4-oxocyclohexa-2,5-dienylidene)hepta-1,4-dienyl]phenolate, and the composition further comprises 5-hydroxy-3-oxo-1-(4-hydroxy-3-methoxyphenyl)-7-(3-methoxy-4-oxocyclohexa-2,5-dienylidene)hepta-1,4-diene (which is the conjugate acid of 2-methoxy-4-[5-hydroxy-3-oxo-7-(3-methoxy-4-oxocyclohexa-2,5-dienylidene)hepta-1,4-dienyl]phenolate).

In some embodiments, the anion is 2-methoxy-4-[5-hydroxy-3-oxo-7-(3-methoxy-4-oxocyclohexa-2,5-dienylidene)hepta-1,5-dienyl]phenolate, and the composition further comprises 5-hydroxy-3-oxo-1-(4-hydroxy-3-methoxyphenyl)-7-(3-methoxy-4-oxocyclohexa-2,5-dienylidene)hepta-1,5-diene (which is the conjugate acid of 2-methoxy-4-[5-hydroxy-3-oxo-7-(3-methoxy-4-oxocyclohexa-2,5-dienylidene)hepta-1,5-dienyl]phenolate).

In some embodiments, the anion is 2-methoxy-4-[3,5-dioxo-7-(3-methoxy-4-oxocyclohexa-2,5-dienylidene)hept-1-en-1-yl]phenolate, and the composition further comprises 3,5-dioxo-1-(4-hydroxy-3-methoxyphenyl)-7-(3-methoxy-4-oxocyclohexa-2,5-dienylidene)hept-1-ene (which is the conjugate acid of 2-methoxy-4-[3,5-dioxo-7-(3-methoxy-4-oxocyclohexa-2,5-dienylidene)hept-1-en-1-yl]phenolate).

In some embodiments, the anion is 2-methoxy-4-[3,5-dihydroxy-7-(3-methoxy-4-oxocyclohexa-2,5-dienylidene)hepta-1,3,5-trienyl]phenolate, and the composition further comprises 3,5-dihydroxy-1-(4-hydroxy-3-methoxyphenyl)-7-(3-methoxy-4-oxocyclohexa-2,5-dienylidene)hepta-1,3,5-triene (which is the conjugate acid of 2-methoxy-4-[3,5-dihydroxy-7-(3-methoxy-4-oxocyclohexa-2,5-dienylidene)hepta-1,3,5-trienyl]phenolate).

In some embodiments, the anion is 5-hydroxy-1-(4-hydroxy-3-methoxyphenyl)-7-(3-methoxy-4-oxocyclohexa-2,5-dienylidene)hepta-1,3,5-triene-3-oxide, and the composition further comprises 3,5-dihydroxy-1-(4-hydroxy-3-methoxyphenyl)-7-(3-methoxy-4-oxocyclohexa-2,5-dienylidene)hepta-1,3,5-triene (which is the conjugate acid of 5-hydroxy-1-(4-hydroxy-3-methoxyphenyl)-7-(3-methoxy-4-oxocyclohexa-2,5-dienylidene)hepta-1,3,5-triene-3-oxide).

In some embodiments, the anion is 3-hydroxy-1-(4-hydroxy-3-methoxyphenyl)-7-(3-methoxy-4-oxocyclohexa-2,5-dienylidene)hepta-1,3,5-triene-5-oxide, and the composition further comprises 3,5-dihydroxy-1-(4-hydroxy-3-methoxyphenyl)-7-(3-methoxy-4-oxocyclohexa-2,5-dienylidene)hepta-1,3,5-triene (which is the conjugate acid of 3-hydroxy-1-(4-hydroxy-3-methoxyphenyl)-7-(3-methoxy-4-oxocyclohexa-2,5-dienylidene)hepta-1,3,5-triene-5-oxide).

In some embodiments, the anion is 5-oxo-1-(4-hydroxy-3-methoxyphenyl)-7-(3-methoxy-4-oxocyclohexa-2,5-dienylidene)hepta-1,3-diene-3-oxide, and the composition further comprises 3-hydroxy-5-oxo-1-(4-hydroxy-3-methoxyphenyl)-7-(3-methoxy-4-oxocyclohexa-2,5-dienylidene)hepta-1,3-diene (which is the conjugate acid of 5-oxo-1-(4-hydroxy-3-methoxyphenyl)-7-(3-methoxy-4-oxocyclohexa-2,5-dienylidene)hepta-1,3-diene-3-oxide).

In some embodiments, the anion is 3-oxo-1-(4-hydroxy-3-methoxyphenyl)-7-(3-methoxy-4-oxocyclohexa-2,5-dienylidene)hepta-1,4-diene-5-oxide, and the composition further comprises 5-hydroxy-3-oxo-1-(4-hydroxy-3-methoxyphenyl)-7-(3-methoxy-4-oxocyclohexa-2,5-dienylidene)hepta-1,4-diene (which is the conjugate acid of 3-oxo-1-(4-hydroxy-3-methoxyphenyl)-7-(3-methoxy-4-oxocyclohexa-2,5-dienylidene)hepta-1,4-diene-5-oxide).

In some embodiments, the anion is 3-oxo-1-(4-hydroxy-3-methoxyphenyl)-7-(3-methoxy-4-oxocyclohexa-2,5-dienylidene)hepta-1,5-diene-5-oxide, and the composition further comprises 5-hydroxy-3-oxo-1-(4-hydroxy-3-methoxyphenyl)-7-(3-methoxy-4-oxocyclohexa-2,5-dienylidene)hepta-1,5-diene (which is the conjugate acid of 3-oxo-1-(4-hydroxy-3-methoxyphenyl)-7-(3-methoxy-4-oxocyclohexa-2,5-dienylidene)hepta-1,5-diene-5-oxide).

In some embodiments, the anion is 3-hydroxy-5-oxo-1-(4-hydroxy-3-methoxyphenyl)-7-(3-methoxy-4-oxocyclohexa-2,5-dienylidene)hepta-1,3-dien-6-ide, and the composition further comprises 3-hydroxy-5-oxo-1-(4-hydroxy-3-methoxyphenyl)-7-(3-methoxy-4-oxocyclohexa-2,5-dienylidene)hepta-1,3-diene (which is the conjugate acid of 3-hydroxy-5-oxo-1-(4-hydroxy-3-methoxyphenyl)-7-(3-methoxy-4-oxocyclohexa-2,5-dienylidene)hepta-1,3-dien-6-ide).

In some embodiments, the anion is 5-hydroxy-3-oxo-1-(4-hydroxy-3-methoxyphenyl)-7-(3-methoxy-4-oxocyclohexa-2,5-dienylidene)hepta-1,4-dien-6-ide, and the composition further comprises 5-hydroxy-3-oxo-1-(4-hydroxy-3-methoxyphenyl)-7-(3-methoxy-4-oxocyclohexa-2,5-dienylidene)hepta-1,4-diene (which is the conjugate acid of 5-hydroxy-3-oxo-1-(4-hydroxy-3-methoxyphenyl)-7-(3-methoxy-4-oxocyclohexa-2,5-dienylidene)hepta-1,4-dien-6-ide).

In some embodiments, the anion is 5-hydroxy-3-oxo-1-(4-hydroxy-3-methoxyphenyl)-7-(3-methoxy-4-oxocyclohexa-2,5-dienylidene)hepta-1,5-dien-4-ide, and the composition further comprises 5-hydroxy-3-oxo-1-(4-hydroxy-3-methoxyphenyl)-7-(3-methoxy-4-oxocyclohexa-2,5-dienylidene)hepta-1,5-diene (which is the conjugate acid of 5-hydroxy-3-oxo-1-(4-hydroxy-3-methoxyphenyl)-7-(3-methoxy-4-oxocyclohexa-2,5-dienylidene)hepta-1,5-dien-4-ide).

In some embodiments, the anion is 3,5-dioxo-1-(4-hydroxy-3-methoxyphenyl)-7-(3-methoxy-4-oxocyclohexa-2,5-dienylidene)hept-1-en-4-ide, and the composition further comprises 3,5-dioxo-1-(4-hydroxy-3-methoxyphenyl)-7-(3-methoxy-4-oxocyclohexa-2,5-dienylidene)hept-1-ene (which is the conjugate acid of 3,5-dioxo-1-(4-hydroxy-3-methoxyphenyl)-7-(3-methoxy-4-oxocyclohexa-2,5-dienylidene)hept-1-en-4-ide).

In some embodiments, the anion is 3,5-dioxo-1-(4-hydroxy-3-methoxyphenyl)-7-(3-methoxy-4-oxocyclohexa-2,5-dienylidene)hept-1-en-6-ide, and the composition further comprises 3,5-dioxo-1-(4-hydroxy-3-methoxyphenyl)-7-(3-methoxy-4-oxocyclohexa-2,5-dienylidene)hept-1-ene (which is the conjugate acid of 3,5-dioxo-1-(4-hydroxy-3-methoxyphenyl)-7-(3-methoxy-4-oxocyclohexa-2,5-dienylidene)hept-1-en-6-ide).

Various aspects of this disclosure relate to a composition, comprising (i) an anion, in which the anion has the chemical structure set forth in either Compound III, which depicts 2-methoxy-4-[3-hydroxy-5-oxo-7-(4-hydroxy-3-methoxyphenyl)hepta-1,3,6-trienyl]phenolate, or Compound IV, which depicts 2-methoxy-4-[5-hydroxy-3-oxo-7-(4-hydroxy-3-methoxyphenyl)hepta-1,4,6-trienyl]phenolate, (ii) a cation at a concentration of at least 10 nanomolar and no greater than 1 molar, in which the cation is a metal cation or an ammonium cation, and (iii) a protic polar solvent at a concentration of at least 5 molar and no greater than 55.5 molar, in which the anion and the cation are solutes that are dissolved in the protic polar solvent.

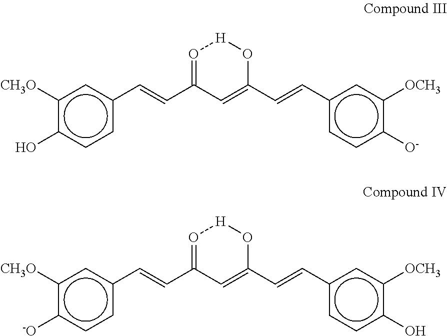

Various aspects of this disclosure relate to a composition, comprising (i) an anion, in which the anion has the chemical structure set forth in Compound V, which depicts 2-methoxy-4-[3,5-dioxo-7-(4-hydroxy-3-methoxyphenyl)hepta-1,6-dienyl]phenolate, (ii) a cation at a concentration of at least 10 nanomolar and no greater than 1 molar, in which the cation is a metal cation or an ammonium cation, and (iii) a protic polar solvent at a concentration of at least 5 molar and no greater than 55.5 molar, in which the anion and the cation are solutes that are dissolved in the protic polar solvent.

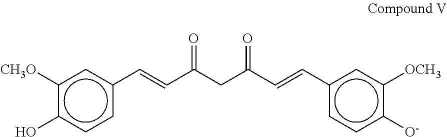

In some embodiments, the composition comprises the anion at a concentration of at least 1 micromolar and no greater than 1 molar. In some specific embodiments, the composition comprises a molecule, in which: the composition comprises the anion at a concentration of at least 1 micromolar and no greater than 1 molar; the anion has a conjugate acid; the molecule is the conjugate acid of the anion; the solutes comprise the molecule; the molecule is dissolved in the protic polar solvent at a concentration of at least 10 nanomolar and no greater than 100 millimolar; and the composition comprises the molecule and the anion at a molar ratio of at least 1:100,000 and no greater than 10:1.

In some embodiments, the composition comprises a Brønsted base, in which: the protic polar solvent has a conjugate base; the Brønsted base is the conjugate base of the protic polar solvent; the solutes comprise the Brønsted base; the Brønsted base is dissolved in the protic polar solvent at a concentration of at least 1 nanomolar and no greater than 5 molar; and the molar concentration of the solvent in the composition is at least 10 times greater than the molar concentration of the Brønsted base that is dissolved in the protic polar solvent. “Brønsted base” refers to a proton acceptor.

In some embodiments, the Brønsted base is either hydroxide; ethoxide; 1-hydroxy-propane-2-oxide; 2-hydroxy-propane-1-oxide; 3-hydroxy-propane-1-oxide; 1,3-dihydroxy-propane-2-oxide; or 2,3-dihydroxy-propane-1-oxide.

In some embodiments, the Brønsted base is hydroxide; and the protic polar solvent is water.

In some embodiments, the Brønsted base is ethoxide; and the protic polar solvent is ethanol.

In some embodiments, the Brønsted base is either 1-hydroxy-propane-2-oxide or 2-hydroxy-propane-1-oxide; and the protic polar solvent is propane-1,2-diol.

In some embodiments, the Brønsted base is 3-hydroxy-propane-1-oxide; and the protic polar solvent is propane-1,3-diol.

In some embodiments, the Brønsted base is either 1,3-dihydroxy-propane-2-oxide or 2,3-dihydroxy-propane-1-oxide; and the protic polar solvent is propane-1,2,3-triol.

In some embodiments, the composition comprises the Brønsted base and the protic polar solvent at a molar ratio of at least 1:1,000,000,000 and no greater than 1:10. In some specific embodiments, the composition comprises the Brønsted base and the protic polar solvent at a molar ratio of at least 1:10,000,000 and no greater than 1:1,000.

In some embodiments, the composition comprises a cosolvent, in which the protic polar solvent and the cosolvent are different chemical species; and the composition comprises the solvent at greater concentration by mass than the cosolvent.

In some embodiments, the cosolvent is either water; ethanol; propane-1,2-diol; propane-1,3-diol; or propane-1,2,3-triol.

In some embodiments, the solvent is water; and the cosolvent is either ethanol, propane-1,2-diol, propane-1,3-diol, or propane-1,2,3-triol. In some specific embodiments, the solvent is water; and the cosolvent is ethanol. In some specific embodiments, the solvent is water; and the cosolvent is propane-1,2-diol. In some specific embodiments, the solvent is water; and the cosolvent is propane-1,3-diol. In some specific embodiments, the solvent is water; and the cosolvent is propane-1,2,3-triol.

In some embodiments, the solvent is ethanol; and the cosolvent is either water, propane-1,2-diol, propane-1,3-diol, or propane-1,2,3-triol. In some specific embodiments, the solvent is ethanol; and the cosolvent is water. In some specific embodiments, the solvent is ethanol; and the cosolvent is propane-1,2-diol. In some specific embodiments, the solvent is ethanol; and the cosolvent is propane-1,3-diol. In some specific embodiments, the solvent is ethanol; and the cosolvent is propane-1,2,3-triol.

In some embodiments, the solvent is either propane-1,2-diol, propane-1,3-diol, or propane-1,2,3-triol; and the cosolvent is either water or ethanol. In some specific embodiments, the solvent is propane-1,2-diol; and the cosolvent is ethanol. In some specific embodiments, the solvent is propane-1,2-diol; and the cosolvent is water. In some specific embodiments, the solvent is propane-1,3-diol; and the cosolvent is ethanol. In some specific embodiments, the solvent is propane-1,3-diol; and the cosolvent is water. In some specific embodiments, the solvent is propane-1,2,3-triol; and the cosolvent is ethanol. In some specific embodiments, the solvent is propane-1,2,3-triol; and the cosolvent is water.

In some embodiments, the composition comprises the solvent and the cosolvent at a ratio of greater than 1:1 and no greater than 10,000:1 by mass.

In some embodiments, the composition comprises a second Brønsted base, in which the cosolvent has a conjugate base; and the second Brønsted base is the conjugate base of the cosolvent.

In some embodiments, the composition comprises a second cosolvent, in which the protic polar solvent, the cosolvent, and the second cosolvent are different chemical species; and the composition comprises the cosolvent at greater concentration by mass than the second cosolvent.

In some embodiments, the second cosolvent is either water; ethanol; propane-1,2-diol; propane-1,3-diol; or propane-1,2,3-triol.

In some embodiments, the solvent is either propane-1,2-diol, propane-1,3-diol, or propane-1,2,3-triol; the cosolvent is ethanol; and the second cosolvent is water. In some specific embodiments, the solvent is propane-1,2-diol; the cosolvent is ethanol; and the second cosolvent is water. In some specific embodiments, the solvent is propane-1,3-diol; the cosolvent is ethanol; and the second cosolvent is water. In some specific embodiments, the solvent is propane-1,2,3-triol; the cosolvent is ethanol; and the second cosolvent is water.

In some embodiments, the solvent is water; the cosolvent is either propane-1,2-diol, propane-1,3-diol, or propane-1,2,3-triol; and the second cosolvent is ethanol. In some specific embodiments, the solvent is water; the cosolvent is propane-1,2-diol; and the second cosolvent is ethanol. In some specific embodiments, the solvent is water; the cosolvent is propane-1,3-diol; and the second cosolvent is ethanol. In some specific embodiments, the solvent is water; the cosolvent is propane-1,2,3-triol; and the second cosolvent is ethanol.

In some embodiments, the solvent is water; the cosolvent is ethanol; and the second cosolvent is either propane-1,2-diol, propane-1,3-diol, or propane-1,2,3-triol. In some specific embodiments, the solvent is water; the cosolvent is ethanol; and the second cosolvent is propane-1,2-diol. In some specific embodiments, the solvent is water; the cosolvent is ethanol; and the second cosolvent is propane-1,3-diol. In some specific embodiments, the solvent is water; the cosolvent is ethanol; and the second cosolvent is propane-1,2,3-triol.

In some embodiments, the solvent is ethanol; the cosolvent is water; and the second cosolvent is either propane-1,2-diol, propane-1,3-diol, or propane-1,2,3-triol. In some specific embodiments, the solvent is ethanol; the cosolvent is water; and the second cosolvent is propane-1,2-diol. In some specific embodiments, the solvent is ethanol; the cosolvent is water; and the second cosolvent is propane-1,3-diol. In some specific embodiments, the solvent is ethanol; the cosolvent is water; and the second cosolvent is propane-1,2,3-triol.

In some embodiments, the composition comprises the solvent and the second cosolvent at a ratio of greater than 10:1 and no greater than 10,000:1 by mass.

In some embodiments, the composition comprises a third Brønsted base, in which the second cosolvent has a conjugate base; and the third Brønsted base is the conjugate base of the second cosolvent.

In some embodiments, the cation is an ammonium cation. In some specific embodiments, the cation is either ammonium (“NH4+”); protonated ethanolamine; choline; protonated sphingosine; protonated lysine; or protonated arginine. In some very specific embodiments, the cation is ammonium. In some very specific embodiments, the cation is protonated ethanolamine. In some very specific embodiments, the cation is choline. In some very specific embodiments, the cation is protonated sphingosine. In some very specific embodiments, the cation is protonated lysine. In some very specific embodiments, the cation is protonated arginine.

In some embodiments, the cation is a metal cation. In some specific embodiments, the cation is either sodium cation (“Na+”); potassium cation (“K+”); magnesium cation (“Mg++”); calcium cation (“Ca++”); zinc cation (“Zn++”); manganese cation (“Mn++”); iron (II) cation (“Fe++”); iron (III) cation (“Fe+++”); copper (I) cation (“Cu+”); or copper (II) cation (“Cu++”). In some very specific embodiments, the cation is sodium cation. In some very specific embodiments, the cation is potassium cation. In some very specific embodiments, the cation is magnesium cation. In some very specific embodiments, the cation is calcium cation. In some very specific embodiments, the cation is zinc cation. In some very specific embodiments, the cation is manganese cation. In some very specific embodiments, the cation is iron (II) cation. In some very specific embodiments, the cation is iron (III) cation. In some very specific embodiments, the cation is copper (I) cation. In some very specific embodiments, the cation is copper (II) cation.

In some embodiments, the composition comprises the anion at a concentration of at least 10 milligrams per liter and no greater than 100 grams per liter. In some specific embodiments, the composition comprises the anion at a concentration of at least 10 milligrams per liter and no greater than 100 milligrams per liter. In some specific embodiments, the composition comprises the anion at a concentration of at least 50 milligrams per liter and no greater than 500 milligrams per liter. In some specific embodiments, the composition comprises the anion at a concentration of at least 100 milligrams per liter and no greater than 1 gram per liter. In some specific embodiments, the composition comprises the anion at a concentration of at least 500 milligrams per liter and no greater than 5 grams per liter. In some specific embodiments, the composition comprises the anion at a concentration of at least 1 gram per liter and no greater than 10 grams per liter. In some specific embodiments, the composition comprises the anion at a concentration of at least 5 grams per liter and no greater than 50 grams per liter. In some specific embodiments, the composition comprises the anion at a concentration of at least 10 grams per liter and no greater than 100 grams per liter. In some specific embodiments, the composition comprises the anion at a concentration of at least 50 grams per liter and no greater than 100 grams per liter.

In some embodiments, the composition has a color, and the color is red.

In some embodiments, the composition lacks either (i) one; or (ii) more than one; or (iii) all of: methanol, ethane-1,2-diol, 2-propanol, 2-methoxyethanol, 1,2-dimethoxyethane, 2-ethoxyethanol, diethyl ether, petroleum ether, tetrahydrofuran, sulfolane, N-methyl-2-pyrrolidone, 1,4-dioxane, pyridine, benzene, cyclohexane, toluene, methylcyclohexane, chlorobenzene, ortho-xylene, meta-xylene, para-xylene, cumene, tetralin, hexane, 2-hexanone, methyl isobutyl ketone, acetonitrile, N,N-dimethylacetamide, N,N-dimethylformamide, formamide, formic acid, formate anion, trifluoroacetic acid, trifluoroacetate anion, 1,1,1-trichloroethane, 1,1,2-trichloroethene, 1,2-dichloroethane, 1,2-dichloroethene, 1,1-dichloroethene, dichloromethane, chloroform, carbon tetrachloride, and nitromethane.

In some embodiments, the composition comprises at least 0.1 and no greater than 6 food calories per gram of the composition. In some specific embodiments, the composition comprises at least 0.1 and no greater than 2 food calories per gram of the composition. In some specific embodiments, the composition comprises at least 1 and no greater than 3 food calories per gram of the composition. In some specific embodiments, the composition comprises at least 2 and no greater than 4 food calories per gram of the composition. In some specific embodiments, the composition comprises at least 3 and no greater than 5 food calories per gram of the composition. In some specific embodiments, the composition comprises at least 4 and no greater than 6 food calories per gram of the composition.

In some embodiments, the composition comprises one or more sugar alcohols.

In some embodiments, the composition comprises one or more sugar alcohols, wherein the one or more sugar alcohols comprise at least 10 percent of the food calories of the composition. In some specific embodiments, the composition comprises one or more sugar alcohols, wherein the one or more sugar alcohols comprise at least 50 percent of the food calories of the composition. In some very specific embodiments, the composition comprises one or more sugar alcohols, wherein the one or more sugar alcohols comprise at least 75 percent of the food calories of the composition.

In some embodiments, each sugar alcohol of the one or more sugar alcohols has the chemical formula C₃H₈O₂, C₃H₈O₃, C₄H₁₀O₂, C₄H₁₀O₄, C₅H₁₂O₅, C₆H₁₂O₆, C₆H₁₄O₆, or C₇H₁₆O₇. In some specific embodiments, the one or more sugar alcohols consist of one or more of 1,2-propanediol; 1,3-propanediol; 1,2,3-propanetriol; 1,3-butanediol; 1,4-butanediol; 2,3-butanediol; butane-1,2,3,4-tetrol; pentane-1,2,3,4,5-pentol; cyclohexane-1,2,3,4,5,6-hexol; hexane-1,2,3,4,5,6-hexol; and heptane-1,2,3,4,5,6,7-heptol. In some even more specific embodiments, the one or more sugar alcohols consist of one or more of propylene glycol, glycerol, erythritol, threitol, arabitol, xylitol, ribitol, mannitol, sorbitol, galactitol, fucitol, iditol, inositol, and volemitol. In some very specific embodiments, the one or more sugar alcohols consist of propylene glycol. In some very specific embodiments, the one or more sugar alcohols consist of glycerol. In some very specific embodiments, the one or more sugar alcohols consist of erythritol. In some very specific embodiments, the one or more sugar alcohols consist of xylitol. In some very specific embodiments, the one or more sugar alcohols consist of sorbitol.

In some embodiments, the one or more sugar alcohols comprise a sugar alcohol that has a conjugate base; and the composition comprises the conjugate base. In some specific embodiments, the one or more sugar alcohols comprise a sugar alcohol that has a conjugate base; and the sugar alcohol and the conjugate base of the sugar alcohol have a molar ratio of at least 10:1 and no greater than 10,000,000:1 in the composition. In some very specific embodiments, the one or more sugar alcohols comprise a sugar alcohol that has a conjugate base; and the sugar alcohol and the conjugate base of the sugar alcohol have a molar ratio of at least 100:1 and no greater than 1,000,000:1 in the composition.

In some embodiments, the composition is a beverage that is suitable for human consumption.

In some embodiments, the composition is an ingredient that is suitable for use in preparing a beverage that is suitable for human consumption.

In some embodiments, the composition is tincture that is suitable for human consumption.

In some embodiments, the composition is an ingredient that is suitable for use in preparing a tincture that is suitable for human consumption.

In some embodiments, the composition is for use as a medicament. Pharmaceutical compositions can be formulated according to REMINGTON: THE SCIENCE AND PRACTICE OF PHARMACY, 22nd edition (Allen Jr, Loyd V., editor) Pharmaceutical Press, 2012.

In some embodiments, the composition is for use in manufacturing a medicament.

In some embodiments, the composition is for use to treat a health condition.

Various aspects of this disclosure relate to a container that contains a composition described anywhere in this disclosure.

In some embodiments, the container is hermetically-sealed.

In some embodiments, the container is a 55-gallon drum or a keg.

In some embodiments, the container is a stainless-steel 55-gallon drum or a stainless-steel keg.

In some embodiments, the container is a plastic bottle, a glass bottle, or an aluminum can.

In some embodiments, the container comprises a label that is either a Nutrition Facts label or a Supplement Facts label. In some specific embodiments, the label states that an ingredient is either “ionized turmeric,” “ionized curcumin,” “turmeric bitters,” or “curcumin bitters.”

In some embodiments, the container contains at least 50 microliters and no greater than 250 liters of the composition. In some embodiments, the container contains at least 75 microliters and no greater than 750 microliters of the composition. In some embodiments, the container contains at least 500 microliters and no greater than 10 milliliters of the composition. In some embodiments, the container contains at least 5 milliliters and no greater than 75 milliliters of the composition. In some embodiments, the container contains at least 50 milliliters and no greater than 150 milliliters of the composition. In some embodiments, the container contains at least 100 milliliters and no greater than 250 milliliters of the composition. In some embodiments, the container contains at least 200 milliliters and no greater than 400 milliliters of the composition. In some embodiments, the container contains at least 300 milliliters and no greater than 600 milliliters of the composition. In some embodiments, the container contains at least 450 milliliters and no greater than 900 milliliters of the composition. In some embodiments, the container contains at least 600 milliliters and no greater than 1200 milliliters of the composition. In some embodiments, the container contains at least 10 liters and no greater than 250 liters of the composition.

Various aspects of this disclosure relate to a method to change the color of a liquid, comprising (i) providing a composition described anywhere in this disclosure, in which the composition has an initial color; and (ii) contacting the composition with a Brønsted acid such that the initial color changes to a final color, in which the initial color and the final color are different.

Various aspects of this disclosure relate to a method to change the color of a liquid, comprising (i) providing a container described anywhere in this disclosure, in which the container contains a composition described anywhere in this disclosure, and the composition has an initial color; and (ii) contacting the composition with a Brønsted acid such that the initial color changes to a final color, in which the initial color and the final color are different.

Various aspects of this disclosure relate to a method to market a consumer product, comprising (i) providing a composition described anywhere in this disclosure, in which the composition has an initial color; and (ii) contacting the composition with a Brønsted acid such that the initial color changes to a final color, in which the initial color and the final color are different.

Various aspects of this disclosure relate to a method to market a consumer product, comprising (i) providing a container described anywhere in this disclosure, in which the container contains a composition described anywhere in this disclosure, and the composition has an initial color; and (ii) contacting the composition with a Brønsted acid such that the initial color changes to a final color, in which the initial color and the final color are different.

In some embodiments, the method further comprises recording a first image of the composition prior to contacting the composition with the Brønsted acid; and recording a second image of the composition after contacting the composition with the Brønsted acid.

In some embodiments, the method further comprises recording video of the composition while the composition is contacted with the Brønsted acid.

In some embodiments, the initial color is red; and the final color is gold.

In some embodiments, the Brønsted acid is citric acid.

In some embodiments, the Brønsted acid is carbonic acid.

In some embodiments, contacting the composition with the Brønsted acid comprises contacting the composition with a carbonated beverage.

In some embodiments, contacting the composition with the Brønsted acid comprises contacting the composition with a Citrus fruit.

In some embodiments, the method further comprises consuming a portion of the composition after contacting the composition with the Brønsted acid, in which a human consumes the portion of the composition by either swallowing or otherwise orally administering it.

In some embodiments, the method further comprises consuming the composition after contacting the composition with the Brønsted acid, in which a human consumes the composition by either swallowing or otherwise orally administering it.

Various aspects of this disclosure relate to a method to administer a composition described anywhere in this disclosure, comprising (i) providing the composition; and (ii) consuming a portion of the composition, in which a human consumes the portion of the composition by either swallowing or otherwise orally administering it.

Various aspects of this disclosure relate to a method to administer a composition described anywhere in this disclosure, comprising (i) providing the composition; and (ii) consuming the composition, in which a human consumes the composition by either swallowing or otherwise orally administering it.

Various aspects of this disclosure relate to a method to administer a composition, comprising (i) providing a container described anywhere in this disclosure; and (ii) consuming a portion of the composition contained within the container, in which a human consumes the portion of the composition by either swallowing or otherwise orally administering it.

Various aspects of this disclosure relate to a method to administer a composition, comprising (i) providing a container described anywhere in this disclosure; and (ii) consuming the composition contained within the container, in which a human consumes the composition by either swallowing or otherwise orally administering it.

In some embodiments, the human has a health condition.

Various aspects of this disclosure relate to a method to treat a health condition, comprising (i) providing a composition described anywhere in this disclosure; and (ii) consuming a therapeutically effective amount of the composition, in which a human consumes the therapeutically effective amount of the composition by either swallowing or otherwise orally administering it.

Various aspects of this disclosure relate to a method to treat a health condition, comprising (i) providing a container described anywhere in this disclosure; and (ii) consuming a therapeutically effective amount of the composition contained within the container, in which a human consumes the therapeutically effective amount of the composition by either swallowing or otherwise orally administering it.

“To treat” refers to at least one of: to cure a health condition; to increase the probability that a health condition will be cured; to shorten the time over which a health condition is cured; to increase the probability that the time necessary to cure a health condition will be shortened; to decrease the severity of a health condition; to increase the probability that the severity of a health condition will decrease; to shorten the time over which the severity of a health condition is decreased; to increase the probability that the time necessary to decrease the severity of a health condition will be shortened; to inhibit a health condition from worsening; to increase the probability that a health condition will not worsen; to delay the worsening of a health condition; to increase the probability that the worsening of a health condition will be delayed; to inhibit the occurrence or recurrence of a health condition; to decrease the probability that a health condition will occur or reoccur; to delay the onset of a health condition; to increase the probability that the onset of a health condition will be delayed; to alleviate at least one symptom of a health condition; to increase the probability that at least one symptom of a health condition will be alleviated; to shorten the time over which at least one symptom of a health condition is alleviated; to increase the probability that the time necessary to alleviate at least one symptom of a health condition will be shortened; to decrease the severity of at least one symptom of a health condition; to increase the probability that the severity of at least one symptom of a health condition will be decreased; to shorten the time over which the severity of at least one symptom of a health condition is decreased; to increase the probability that the time necessary to decrease the severity of at least one symptom of a health condition will be shortened; to inhibit at least one symptom of a health condition from worsening; to increase the probability that at least one symptom of a health condition will not worsen; to delay the worsening of at least one symptom of a health condition; to increase the probability that the worsening of at least one symptom of a health condition will be delayed; to inhibit at least one symptom of a health condition from occurring or reoccurring; to decrease the probability that at least one symptom of a health condition will occur or reoccur; to delay the onset of at least one symptom of a health condition; and to increase the probability that the onset of at least one symptom of a health condition will be delayed.

In some embodiments, the health condition is cancer.

In some embodiments, the health condition is a carcinoma, a sarcoma, a lymphoma, a leukemia, a germ cell tumor, a blastoma.

In some embodiments, the health condition is brain cancer.

In some embodiments, the health condition is ovarian cancer.

In some embodiments, the health condition is breast cancer.

In some embodiments, the health condition is vaginal cancer.

In some embodiments, the health condition is vulvar cancer.

In some embodiments, the health condition is uterine cancer.

In some embodiments, the health condition is cervical cancer.

In some embodiments, the health condition is endometrial cancer.

In some embodiments, the health condition is prostate cancer.

In some embodiments, the health condition is testicular cancer.

In some embodiments, the health condition is penile cancer.

In some embodiments, the health condition is liver cancer.

In some embodiments, the health condition is intrahepatic bile duct cancer.

In some embodiments, the health condition is lung cancer.

In some embodiments, the health condition is small cell lung cancer.

In some embodiments, the health condition is non-small cell lung cancer.

In some embodiments, the health condition is bronchial cancer.

In some embodiments, the health condition is mesothelioma.

In some embodiments, the health condition is pancreatic cancer.

In some embodiments, the health condition is gall bladder cancer.

In some embodiments, the health condition is non-melanoma skin cancer.

In some embodiments, the health condition is melanoma.

In some embodiments, the health condition is Kaposi sarcoma.

In some embodiments, the health condition is thyroid cancer.

In some embodiments, the health condition is head and neck cancer.

In some embodiments, the health condition is nasopharyngeal cancer.

In some embodiments, the health condition is oropharyngeal cancer.

In some embodiments, the health condition is hypopharyngeal cancer.

In some embodiments, the health condition is laryngeal cancer.

In some embodiments, the health condition is oral cavity cancer.

In some embodiments, the health condition is tongue cancer.

In some embodiments, the health condition is mouth cancer.

In some embodiments, the health condition is salivary gland cancer.

In some embodiments, the health condition is esophageal cancer.

In some embodiments, the health condition is gastric cancer.

In some embodiments, the health condition is colorectal cancer.

In some embodiments, the health condition is colon cancer.

In some embodiments, the health condition is rectal cancer.

In some embodiments, the health condition is anal cancer.

In some embodiments, the health condition is kidney cancer.

In some embodiments, the health condition is renal cell cancer.

In some embodiments, the health condition is renal pelvis cancer.

In some embodiments, the health condition is bladder cancer.

In some embodiments, the health condition is urethral cancer.

In some embodiments, the health condition is Hodgkin lymphoma.

In some embodiments, the health condition is non-Hodgkin's lymphoma.

In some embodiments, the health condition is myeloma.

In some embodiments, the health condition is multiple myeloma.

In some embodiments, the health condition is acute lymphocytic leukemia.

In some embodiments, the health condition is chronic lymphocytic leukemia.

In some embodiments, the health condition is acute myeloid leukemia.

In some embodiments, the health condition is chronic myeloid leukemia.

In some embodiments, the health condition is osteosarcoma.

In some embodiments, the health condition is a soft tissue cancer.

In some embodiments, the health condition is a viral infection or a bacterial infection.

In some embodiments, the health condition is a viral infection caused by an enveloped virus. In some specific embodiments, the health condition is an influenza virus infection. In some specific embodiments, the health condition is a coronavirus infection. In some specific embodiments, the health condition is a pneumococcal infection. In some specific embodiments, the health condition is a human immunodeficiency virus (“HIV”) infection. In some specific embodiments, the health condition is a hepatitis delta virus infection. In some specific embodiments, the health condition is a Zika virus infection. In some specific embodiments, the health condition is an Ebola virus infection.

In some embodiments, the health condition is a bacterial infection. In some specific embodiments, the health condition is a Clostridium difficile infection. In some specific embodiments, the health condition is a Staphylococcus aureus infection. In some specific embodiments, the health condition is a Mycobacterium tuberculosis infection. In some specific embodiments, the health condition is an Enterococcus faecalis infection. In some specific embodiments, the health condition is a Salmonella infection.

In some embodiments, the health condition is an autoimmune disease. In some specific embodiments, the health condition is arthritis. In some specific embodiments, the health condition is inflammatory bowel disease. In some specific embodiments, the health condition is psoriasis. In some specific embodiments, the health condition is lupus. In some specific embodiments, the health condition is multiple sclerosis.

In some embodiments, the health condition is a neurodegenerative disease. In some specific embodiments, the health condition has a progression that correlates with increasing amyloid. In some specific embodiments, the health condition is Alzheimer's Disease. In some specific embodiments, the health condition is Parkinson's Disease. In some specific embodiments, the health condition is transmissible spongiform encephalopathy. In some specific embodiments, the health condition is tauopathy. In some specific embodiments, the health condition is Huntington's Disease. In some specific embodiments, the health condition is amyloidosis.

In some embodiments, the health condition is type II diabetes.

In some embodiments, the health condition is myocardial infarction.

In some embodiments, the health condition is ischemic stroke.

In some embodiments, the health condition is Sickle cell disease.

In some embodiments, the health condition is hyperlipidemia.

In some embodiments, the health condition is hypercholesterolemia.

In some embodiments, providing a composition consists of obtaining ownership of the composition. In some specific embodiments, providing a composition consists of purchasing the composition.

In some embodiments, providing a container consists of obtaining ownership of the container. In some specific embodiments, providing a container consists of purchasing the container.

In some embodiments, providing a composition consists of obtaining physical possession of the composition.

In some embodiments, providing a container consists of obtaining physical possession of the container.

In some embodiments, providing a composition consists of obtaining physical access to the composition.

In some embodiments, providing a container consists of obtaining physical access to the container.

In some embodiments, providing a composition consists of opening a container that contains the composition.

In some embodiments, providing a container consists of unpackaging the container.

In some embodiments, providing a composition consists of formulating or otherwise manufacturing the composition.

In some embodiments, providing a container consists of transferring a composition into a starting container to produce the container. In some specific embodiments, providing a container consists of transferring a composition into a drinking cup or drinking glass to produce the container.

In some embodiments, providing a composition consists of transferring the composition into a measuring device. In some specific embodiments, providing a composition consists of transferring the composition into a medicine dropper. In some embodiments, providing a composition consists of measuring an amount of a bulk composition to provide the composition.

In some embodiments, providing a composition consists of dispensing an amount of a bulk composition to provide the composition.

The following examples provide a framework to implement certain aspects of the disclosure, and these examples do not limit the scope of this disclosure or any claim that matures from this disclosure.

### EXEMPLIFICATION

### Example 1. Development of Tinctures and Beverages that Contain Conjugate Bases of Various Constitutional Isomers of Curcumin

4.615 grams of potassium hydroxide was dissolved in 300 milliliters of 190 proof ethanol. 30 grams of curcumin was added to the potassium hydroxide in ethanol, and the resultant solution was stirred at room temperature for 30 minutes. 900 milliliters of glycerol was then added to the solution to produce a glycerol tincture.

200 microliter doses of the tincture were administered orally to consenting adults and produced favorable effects. The favorable effects had an onset time of approximately 1 minute and a duration of approximately 3 hours.

The tincture had a red color, and it was water miscible. After mixing the tincture with water, the addition of carbonated water (which contains carbonic acid) converted the anions of the tincture into their conjugate acids as evidenced by a color change from red to gold. Water containing either the anions or their conjugate acids displayed both favorable flavors and favorable effects. 1 milliliter of the tincture was also added to a serving of Coors Light beer (Coors Brewing Company, Golden, Colorado) and a serving of White Rascal beer (Avery Brewing Co, Boulder, Colorado), and both combinations resulted in favorable flavors and favorable effects.

Curcumin is consumed in large quantities in curries and other foods that contain turmeric, but the oral consumption of curcumin is not known to display any perceptible effect. The 1-minute onset time of a perceptible effect strongly suggests that the conjugate acids of various stereoisomers of curcumin are absorbed through the lining of the mouth and esophagus, which significantly improved bioavailability.

### Example 2. Development of Bitters Products for Use in Beverages

1.828 grams of potassium hydroxide was dissolved in 120 milliliters of 190 proof ethanol. 12 grams of curcumin was added to the potassium hydroxide in ethanol, and the resultant solution was stirred at room temperature for 30 minutes. 96 milliliters of 190 proof ethanol was thoroughly mixed with the solution, and then 264 milliliters of glycerol was thoroughly mixed with the solution to produce bitters.

The bitters product had a red color, and it was water miscible. After mixing the bitters with water, the addition of carbonated water (which contains carbonic acid) converted the anions of the bitters into their conjugate acids as evidenced by a color change from red to gold. Water containing either the anions or their conjugate acids displayed both favorable flavors and favorable effects.

Curcumin is insoluble in water, which limits its bioavailability. The development of water-miscible bitters using anionic forms of both curcumin and its constitutional isomers significantly increased solubility, which may partially account for the significantly increased bioavailability described in Example 1. The literature value for the solubility of curcumin in ethanol is 10 milligrams per milliliter, and Examples 1 & 2 clearly demonstrate a 10-fold increase in solubility.

### Example 3. Development of Beverages

Bitters were made using a strategy similar to those described in Example 2. The bitters were added to 500 milliliter bottles of ESSENTIA® OVERACHIEVING H₂O®, which contains purified water, sodium bicarbonate, dipotassium phosphate, magnesium sulfate, and calcium chloride, and which has a pH of about 9.5, such that each bottle contained 100 milligrams of dissolved anions derived from curcumin. Bottles were stored at various temperatures ranging from −18 degrees Celsius to 65 degrees Celsius for stability testing, and temperature did not appear to result in precipitation or otherwise cause any instability. This Example 3 therefore demonstrates that the methods described in this disclosure can be used to produce aqueous liquids containing anions of various constitutional isomers of curcumin that can be dissolved in water at high concentrations relative to conventional curcumin, which is insoluble in water.

What is claimed is:

1. A composition, comprising:
an anion, wherein the anion has the chemical formula C₂₁H₁₉O₆¹⁻; the anion has the chemical structure set forth in Compound XXX; Compound XXX contains exactly 10 double bonds; R1 is hydroxy, oxide, or oxo; R2 is hydroxy, oxide, or oxo; R3 is hydroxy or oxide; and the 3 dotted lines in Compound XXX depict 0, 1, or 2 double bonds; 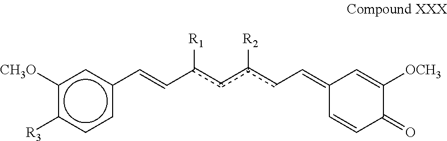
a cation at a concentration of at least 10 nanomolar and no greater than 1 molar, wherein the cation is a metal cation or an ammonium cation; and
a protic polar solvent at a concentration of at least 5 molar and no greater than 55.5 molar, wherein the anion and the cation are solutes that are dissolved in the protic polar solvent.

2. The composition of claim 1, wherein the anion is either:
2-methozxy-4-[3-hydroxy-5-oxo-7-(3-methoxy-4-oxocyclohexa-2,5-dienylidene)hepta-1,3-dienyl]phenolate;
2-methoxy-4-[5-hydroxy-3-oxo-7-(3-methoxy-4-oxocyclohexa-2,5-dienylidene)hepta-1,4-dienyl]phenolate;
2-methoxy-4-[5-hydroxy-3-oxo-7-(3-methoxy-4-oxocyclohexa-2,5-dienylidene)hepta-1,5-dienyl]phenolate;
2-methoxy-4-[3,5-dioxo-7-(3-methoxy-4-oxocyclohexa-2,5-dienylidene)hept-1-en-1-yl]phenolate;
2-methoxy-4-[3,5-dihydroxy-7-(3-methoxy-4-oxocyclohexa-2,5-dienylidene)hepta-1,3,5-trienyl]phenolate;
5-hydroxy-1-(4-hydroxy-3-methoxyphenyl)-7-(3-methoxy-4-oxocyclohexa-2,5-dienylidene)hepta-1,3,5-triene-3-oxide;
3-hydroxy-1-(4-hydroxy-3-methoxyphenyl)-7-(3-methoxy-4-oxocyclohexa-2,5-dienylidene)hepta-1,3,5-triene-5-oxide;
5-oxo-1-(4-hydroxy-3-methoxyphenyl)-7-(3-methoxy-4-oxocyclohexa-2,5-dienylidene)hepta-1,3-diene-3-oxide;
3-oxo-1-(4-hydroxy-3-methoxyphenyl)-7-(3-methoxy-4-oxocyclohexa-2,5-dienylidene)hepta-1,4-diene-5-oxide;
3-oxo-1-(4-hydroxy-3-methoxyphenyl)-7-(3-methoxy-4-oxocyclohexa-2,5-dienylidene)hepta-1,5-diene-5-oxide;
3-hydroxy-5-oxo-1-(4-hydroxy-3-methoxyphenyl)-7-(3-methoxy-4-oxocyclohexa-2,5-dienylidene)hepta-1,3-dien-6-ide;
5-hydroxy-3-oxo-1-(4-hydroxy-3-methoxyphenyl)-7-(3-methoxy-4-oxocyclohexa-2,5-dienylidene)hepta-1,4-dien-6-ide;
5-hydroxy-3-oxo-1-(4-hydroxy-3-methoxyphenyl)-7-(3-methoxy-4-oxocyclohexa-2,5-dienylidene)hepta-1,5-dien-4-ide;
3,5-dioxo-1-(4-hydroxy-3-methoxyphenyl)-7-(3-methoxy-4-oxocyclohexa-2,5-dienylidene)hept-1-en-4-ide; or
3,5-dioxo-1-(4-hydroxy-3-methoxyphenyl)-7-(3-methoxy-4-oxocyclohexa-2,5-dienylidene)hept-1-en-6-ide.

3. The composition of claim 1, wherein either:
the anion is 2-methoxy-4-[3-hydroxy-5-oxo-7-(3-methoxy-4-oxocyclohexa-2,5-dienylidene)hepta-1,3-dienyl]phenolate, and the composition further comprises 3-hydroxy-5-oxo-1-(4-hydroxy-3-methoxyphenyl)-7-(3-methoxy-4-oxocyclohexa-2,5-dienylidene)hepta-1,3-diene;
the anion is 2-methoxy-4-[5-hydroxy-3-oxo-7-(3-methoxy-4-oxocyclohexa-2,5-dienylidene)hepta-1,4-dienyl]phenolate, and the composition further comprises 5-hydroxy-3-oxo-1-(4-hydroxy-3-methoxyphenyl)-7-(3-methoxy-4-oxocyclohexa-2,5-dienylidene)hepta-1,4-diene;
the anion is 2-methoxy-4-[5-hydroxy-3-oxo-7-(3-methoxy-4-oxocyclohexa-2,5-dienylidene)hepta-1,5-dienyl]phenolate, and the composition further comprises 5-hydroxy-3-oxo-1-(4-hydroxy-3-methoxyphenyl)-7-(3-methoxy-4-oxocyclohexa-2,5-dienylidene)hepta-1,5-diene;
the anion is 2-methoxy-4-[3,5-dioxo-7-(3-methoxy-4-oxocyclohexa-2,5-dienylidene)hept-1-en-1-yl]phenolate, and the composition further comprises 3,5-dioxo-1-(4-hydroxy-3-methoxyphenyl)-7-(3-methoxy-4-oxocyclohexa-2,5-dienylidene)hept-1-ene;
the anion is 2-methoxy-4-[3,5-dihydroxy-7-(3-methoxy-4-oxocyclohexa-2,5-dienylidene)hepta-1,3,5-trienyl]phenolate, and the composition further comprises 3,5-dihydroxy-1-(4-hydroxy-3-methoxyphenyl)-7-(3-methoxy-4-oxocyclohexa-2,5-dienylidene)hepta-1,3,5-triene;
the anion is 5-hydroxy-1-(4-hydroxy-3-methoxyphenyl)-7-(3-methoxy-4-oxocyclohexa-2,5-dienylidene)hepta-1,3,5-triene-3-oxide, and the composition further comprises 3,5-dihydroxy-1-(4-hydroxy-3-methoxyphenyl)-7-(3-methoxy-4-oxocyclohexa-2,5-dienylidene)hepta-1,3,5-triene;
the anion is 3-hydroxy-1-(4-hydroxy-3-methoxyphenyl)-7-(3-methoxy-4-oxocyclohexa-2,5-dienylidene)hepta-1,3,5-triene-5-oxide, and the composition further comprises 3,5-dihydroxy-1-(4-hydroxy-3-methoxyphenyl)-7-(3-methoxy-4-oxocyclohexa-2,5-dienylidene)hepta-1,3,5-triene;
the anion is 5-oxo-1-(4-hydroxy-3-methoxyphenyl)-7-(3-methoxy-4-oxocyclohexa-2,5-dienylidene)hepta-1,3-diene-3-oxide, and the composition further comprises 3-hydroxy-5-oxo-1-(4-hydroxy-3-methoxyphenyl)-7-(3-methoxy-4-oxocyclohexa-2,5-dienylidene)hepta-1,3-diene;
the anion is 3-oxo-1-(4-hydroxy-3-methoxyphenyl)-7-(3-methoxy-4-oxocyclohexa-2,5-dienylidene)hepta-1,4-diene-5-oxide, and the composition further comprises 5-hydroxy-3-oxo-1-(4-hydroxy-3-methoxyphenyl)-7-(3-methoxy-4-oxocyclohexa-2,5-dienylidene)hepta-1,4-diene;
the anion is 3-oxo-1-(4-hydroxy-3-methoxyphenyl)-7-(3-methoxy-4-oxocyclohexa-2,5-dienylidene)hepta-1,5-diene-5-oxide, and the composition further comprises 5-hydroxy-3-oxo-1-(4-hydroxy-3-methoxyphenyl)-7-(3-methoxy-4-oxocyclohexa-2,5-dienylidene)hepta-1,5-diene;
the anion is 3-hydroxy-5-oxo-1-(4-hydroxy-3-methoxyphenyl)-7-(3-methoxy-4-oxocyclohexa-2,5-dienylidene)hepta-1,3-dien-6-ide, and the composition further comprises 3-hydroxy-5-oxo-1-(4-hydroxy-3-methoxyphenyl)-7-(3-methoxy-4-oxocyclohexa-2,5-dienylidene)hepta-1,3-diene;
the anion is 5-hydroxy-3-oxo-1-(4-hydroxy-3-methoxyphenyl)-7-(3-methoxy-4-oxocyclohexa-2,5-dienylidene)hepta-1,4-dien-6-ide, and the composition further comprises 5-hydroxy-3-oxo-1-(4-hydroxy-3-methoxyphenyl)-7-(3-methoxy-4-oxocyclohexa-2,5-dienylidene)hepta-1,4-diene;
the anion is 5-hydroxy-3-oxo-1-(4-hydroxy-3-methoxyphenyl)-7-(3-methoxy-4-oxocyclohexa-2,5-dienylidene)hepta-1,5-dien-4-ide, and the composition further comprises 5-hydroxy-3-oxo-1-(4-hydroxy-3-methoxyphenyl)-7-(3-methoxy-4-oxocyclohexa-2,5-dienylidene)hepta-1,5-diene;
the anion is 3,5-dioxo-1-(4-hydroxy-3-methoxyphenyl)-7-(3-methoxy-4-oxocyclohexa-2,5-dienylidene)hept-1-en-4-ide, and the composition further comprises 3,5-dioxo-1-(4-hydroxy-3-methoxyphenyl)-7-(3-methoxy-4-oxocyclohexa-2,5-dienylidene)hept-1-ene; or
the anion is 3,5-dioxo-1-(4-hydroxy-3-methoxyphenyl)-7-(3-methoxy-4-oxocyclohexa-2,5-dienylidene)hept-1-en-6-ide, and the composition further comprises 3,5-dioxo-1-(4-hydroxy-3-methoxyphenyl)-7-(3-methoxy-4-oxocyclohexa-2,5-dienylidene)hept-1-ene.

4. A composition, comprising:
an anion, wherein the anion has the chemical structure set forth in either Compound III, which depicts 2-methoxy-4-[3-hydroxy-5-oxo-7-(4-hydroxy-3-methoxyphenyl)hepta-1,3,6-trienyl]phenolate, or Compound IV, which depicts 2-methoxy-4-[5-hydroxy-3-oxo-7-(4-hydroxy-3-methoxyphenyl)hepta-1,4,6-trienyl]phenolate; 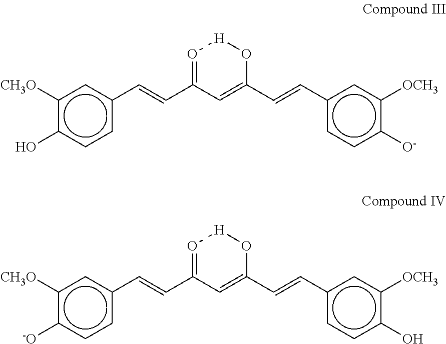
a cation at a concentration of at least 10 nanomolar and no greater than 1 molar, wherein the cation is a metal cation or an ammonium cation; and
a protic polar solvent at a concentration of at least 5 molar and no greater than 55.5 molar, wherein the anion and the cation are solutes that are dissolved in the protic polar solvent.

5. A composition, comprising:
an anion, wherein the anion has the chemical structure set forth in Compound V, which depicts 2-methoxy-4-[3,5-dioxo-7-(4-hydroxy-3-methoxyphenyl)hepta-1,6-dienyl]phenolate; 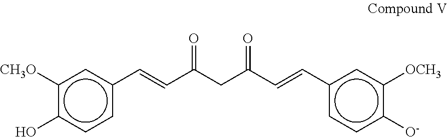
a cation at a concentration of at least 10 nanomolar and no greater than 1 molar, wherein the cation is a metal cation or an ammonium cation; and
a protic polar solvent at a concentration of at least 5 molar and no greater than 55.5 molar, wherein the anion and the cation are solutes that are dissolved in the protic polar solvent.

6. A container that contains a composition according to claim 4.

7. A method to change the color of a liquid, comprising:
providing the composition of claim 4, wherein the composition has an initial color; and
contacting the composition with a Brønsted acid such that the initial color changes to a final color.

8. A method to administer the composition of claim 4, comprising:
providing the composition; and
consuming a portion of the composition, wherein a human consumes the portion of the composition by either swallowing or otherwise orally administering it.

9. The composition of claim 4, comprising a molecule, wherein:
the composition comprises the anion at a concentration of at least 1 micromolar and no greater than 1 molar;
the anion has a conjugate acid;
the molecule is the conjugate acid of the anion;
the solutes comprise the molecule;
the molecule is dissolved in the protic polar solvent at a concentration of at least 10 nanomolar and no greater than 100 millimolar; and
the composition comprises the molecule and the anion at a molar ratio of at least 1:100,000 and no greater than 10:1.

10. The composition of claim 4, comprising a Brønsted base, wherein:
the protic polar solvent has a conjugate base;
the Brønsted base is the conjugate base of the protic polar solvent;
the solutes comprise the Brønsted base;
the Brønsted base is dissolved in the protic polar solvent at a concentration of at least 1 nanomolar and no greater than 5 molar; and
the molar concentration of the solvent in the composition is at least 10 times greater than the molar concentration of the Brønsted base that is dissolved in the protic polar solvent.

11. The composition of claim 4, comprising a Brønsted base, wherein:
the protic polar solvent has a conjugate base;
the Brønsted base is the conjugate base of the protic polar solvent;
the solutes comprise the Brønsted base;
the Brønsted base is dissolved in the protic polar solvent at a concentration of at least 1 nanomolar and no greater than 5 molar;
the molar concentration of the solvent in the composition is at least 10 times greater than the molar concentration of the Brønsted base that is dissolved in the protic polar solvent; and
the Brønsted base is selected from hydroxide; ethoxide; 1-hydroxy-propane-2-oxide; 2-hydroxy-propane-1-oxide; 3-hydroxy-propane-1-oxide; 1,3-dihydroxy-propane-2-oxide; and 2,3-dihydroxy-propane-1-oxide.

12. The composition of claim 4, comprising a cosolvent and a second cosolvent, wherein:
the protic polar solvent, the cosolvent, and the second cosolvent are different chemical species;
the composition comprises the solvent at greater concentration by mass than the cosolvent;
the composition comprises the cosolvent at greater concentration by mass than the second cosolvent;
the solvent is selected from propane-1,2-diol, propane-1,3-diol, and propane-1,2,3-triol;
the cosolvent is ethanol; and
the second cosolvent is water.

13. The composition of claim 4, comprising a cosolvent and a second cosolvent, wherein:
the protic polar solvent, the cosolvent, and the second cosolvent are different chemical species;
the composition comprises the solvent at greater concentration by mass than the cosolvent;
the composition comprises the cosolvent at greater concentration by mass than the second cosolvent;
the solvent is water;
the cosolvent is selected from propane-1,2-diol, propane-1,3-diol, and propane-1,2,3-triol; and
the second cosolvent is ethanol.

14. The composition of claim 4, comprising a cosolvent and a second cosolvent, wherein:
the protic polar solvent, the cosolvent, and the second cosolvent are different chemical species;
the composition comprises the solvent at greater concentration by mass than the cosolvent;
the composition comprises the cosolvent at greater concentration by mass than the second cosolvent;
the solvent is water;
the cosolvent is ethanol; and
the second cosolvent selected from either propane-1,2-diol, propane-1,3-diol, and propane-1,2,3-triol.

15. The composition of claim 4, comprising a cosolvent and a second cosolvent, wherein:
the protic polar solvent, the cosolvent, and the second cosolvent are different chemical species;
the composition comprises the solvent at greater concentration by mass than the cosolvent;
the composition comprises the cosolvent at greater concentration by mass than the second cosolvent;
the solvent is ethanol;
the cosolvent is water; and
the second cosolvent is selected from propane-1,2-diol, propane-1,3-diol, and propane-1,2,3-triol.

16. The composition of claim 4, comprising a cosolvent, wherein:
the protic polar solvent and the cosolvent are different chemical species;
the composition comprises the solvent at greater concentration by mass than the cosolvent;
the solvent is water or ethanol; and
the cosolvent is selected from propane-1,2-diol, propane-1,3-diol, and propane-1,2,3-triol.

17. The composition of claim 4, comprising a cosolvent, wherein:
the protic polar solvent and the cosolvent are different chemical species;
the composition comprises the solvent at greater concentration by mass than the cosolvent;
the solvent is selected from propane-1,2-diol, propane-1,3-diol, and propane-1,2,3-triol; and
the cosolvent is either water or ethanol.

18. The composition of claim 4, wherein the cation is selected from ammonium (“NH4+”); protonated ethanolamine; choline; protonated sphingosine; protonated lysine;
and protonated arginine.

19. The composition of claim 4, wherein the cation is selected from sodium cation (“Na+”); potassium cation (“K+”); magnesium cation (“Mg++”); calcium cation (“Ca++”); zinc cation (“Zn++”); manganese cation (“Mn++”); iron (II) cation (“Fe++”); iron (III) cation (“Fe+++”); copper (I) cation (“Cu+”); and copper (II) cation (“Cu++”).

20. The composition of claim 4, comprising propylene glycol or glycerol.
